(19) United States
(12) Patent Application Publication
(10) Pub. No.: US 2026/0090324 A1
(43) Pub. Date: Mar. 26, 2026

(54) WAFER TRANSFER DEVICE DETECTION SYSTEM AND WAFER TRANSFER DEVICE

(71) Applicant: NANYA TECHNOLOGY CORPORATION, New Taipei City (TW)

(72) Inventor: Yi-Wen LIU, New Taipei City (TW)

(21) Appl. No.: 18/892,557

(22) Filed: Sep. 23, 2024

(51) Int. Cl. H01L 21/67; G01N 33/00; H01L 21/673

(52) U.S. Cl. CPC H10P 72/0616; G01N 33/0004; H10P 72/1924

(57) ABSTRACT

A wafer transfer device detection system includes detectors, a filling device, a wafer transfer device and a control center. Detectors are disposed between a plurality of process equipment in a process. Filling device performs a filling procedure. Wafer transfer device includes body and sensor disposed in body. Wafer transfer device transmits wafer of process. Sensor detects a gas concentration inside wafer transfer device during a transportation stage of the process. If gas concentration is higher than a preset gas concentration, sensor generates a wireless communication signal. Control center is coupled to detectors and filling device. If detectors receive the wireless communication signal. Detectors notify control center so that the control center transmits wafer transfer device to filling device from process. Control center controls the filling device to perform filling procedure so as to adjust gas concentration to a target gas concentration to retransmit wafer transfer device back to process.

### BACKGROUND

### Field of Invention

The present disclosure relates to an electronic system and an electronic device. More particularly, the present disclosure relates to a wafer transfer device detection system and a wafer transfer device.

### Description of Related Art

From a beginning of production to packaging and shipment of conventional wafers in the production line, wafers are transported by conventional wafer transport boxes (e.g.: front opening unified pods (FOUP) and front opening shipping boxes (FOSB)). In a semiconductor manufacturing process, there are strict requirements for moisture or airborne molecular contamination in wafer transport boxes, which must be controlled within a certain range so as not to affect wafers. A current practice is to fill wafer transport boxes with inert gas (nitrogen) to reduce oxidation condition in wafer transport boxes and maintain a dry and stable space of wafer transport boxes.

However, materials used in multiple wafer transport boxes will deteriorate over time or wafer transport boxes have other factors that cause inert gas to escape early. An environment inside a wafer transport box cannot be effectively maintained stable, thereby affecting a yield of wafers or even causing wafers to be scrapped.

For the foregoing reason, there is a need to provide a wafer transfer device detection system and a wafer transfer device to solve the problems of the prior art.

### SUMMARY

One aspect of the present disclosure provides a wafer transfer device detection system. The wafer transfer device detection system includes a plurality of detectors, a filling device, a wafer transfer device and a control center. The detectors are disposed between a plurality of process equipment in a process. The filling device is configured to perform a filling procedure. The wafer transfer device includes a body and a sensor disposed in the body. The wafer transfer device is configured to transmit a wafer of the process. The sensor is configured to detect a gas concentration inside the wafer transfer device during a transportation stage of the process. If the gas concentration is higher than a preset gas concentration, the sensor is configured to generate a wireless communication signal. If the detectors are configured to receive the wireless communication signal, the detectors are configured to notify the control center so that the control center is configured to transmit the wafer transfer device to the filling device from the process. The control center is configured to control the filling device to perform the filling procedure so as to adjust the gas concentration to a target gas concentration to retransmit the wafer transfer device back to the process.

Another aspect of the present disclosure provides a wafer transfer device. The wafer transfer device is configured to transmit a wafer of a process. The wafer transfer device includes a body and a sensor. The body includes a medial wall, a bottom wall, a first opening, a first accommodation space and a second accommodation space. The first opening is relative to the bottom wall. The first accommodation space is jointly defined by the medial wall, the bottom wall and the first opening. The first accommodation space is configured to accommodate the wafer and a wafer holder. The second accommodation space is disposed in one of the medial wall and the medial wall. The second accommodation space includes a second opening. The sensor is disposed in the second accommodation space, and is configured to detect a gas concentration of the first accommodation space and the second accommodation space during a transportation stage of the process. If the gas concentration is higher than a preset gas concentration, the sensor is configured to generate a wireless communication signal to notify a control center to transmit the wafer transfer device to a filling device so that the filling device is configured to perform a filling procedure so as to adjust the gas concentration to a target gas concentration.

In view of the aforementioned shortcomings and deficiencies of the prior art, the present disclosure provides a wafer transfer device detection system and a wafer transfer device so that a gas concentration (e.g.: a gas molecular pollutant concentration and humidity) inside a wafer transfer device can be visualized, and a gas concentration of a wafer transfer device can be immediately adjusted to an appropriate gas concentration to maintain a stability of an internal environment of a transfer device at all times and is not affected by aging of a wafer transfer device or other factors.

### BRIEF DESCRIPTION OF THE DRAWINGS

The present disclosure can be more fully understood by reading the following detailed description of the embodiment, with reference made to the accompanying drawings as follows:

FIG. 1 depicts a schematic diagram of a wafer transfer device detection system according to some embodiments of the present disclosure;

FIG. 2 depicts a schematic diagram of a wafer transfer device according to some embodiments of the present disclosure;

FIG. 3 depicts a schematic diagram of a structure of a wafer transfer device according to some embodiments of the present disclosure;

FIG. 4 depicts a schematic diagram of an external bottom view of a wafer transfer device according to some embodiments of the present disclosure; and

FIG. 5 depicts a schematic flow diagram of a process according to some embodiments of the present disclosure.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

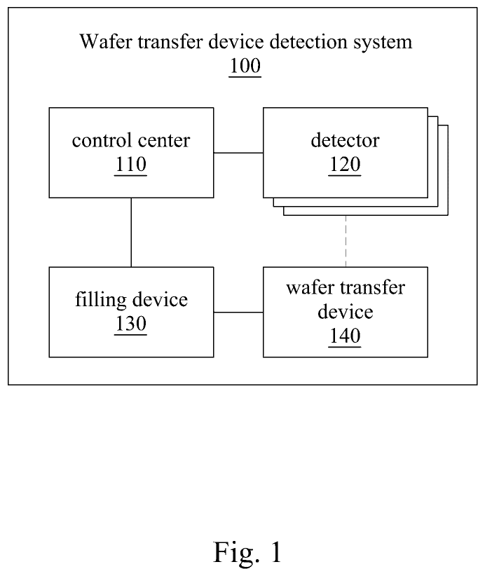

FIG. 1 depicts a schematic diagram of a wafer transfer device detection system 100 according to some embodiments of the present disclosure. The wafer transfer device detection system 100 includes a control center 110, a plurality of detectors 120, a filling device 130 and a wafer transfer device 140. The control center 110 is coupled to the detectors 120 and the filling device 130. A location of each of the detectors 120 and the filling device 130 will be introduced in following paragraphs.

In some embodiments, the control center 110 is a hub of systems such as a production (e.g.: a manufacturing execution systems (MES) and an equipment automation program (EAP)) and transportation systems (e.g.: a material control system (MCS)). The control center 110 is configured obtain various information and statuses such as real-time processes, a position of wafer transport boxes, machines, and transportation commands. Through a series of calculation rules and scheduling plans, the control center 110 is configured to dispatch wafer transport boxes to various stations or process sites in a process in a timely and appropriate manner.

In some embodiments, the detectors 120 are configured to detect wireless communication signals from the wafer transfer device 140 of the present disclosure. The detectors 120 include ultra-high frequency (UHF) radio frequency identification (RFID) readers. A type of the detectors 120 include fixed RFID readers, handheld RFID readers and a production line internet of things (IoT) industrial control RFID readers. Ultra-high frequency (UHF) RFID is of particular interest in the commercial sector because it provides a large read range with low-cost RFID tags and allows organizations to tag large quantities of goods and assets and automatically track them. UHF passive tags have a read range of up to 300 meters.

The filling device 130 is configured to perform a filling process. The filling process is performed by the filling device 130 to fill the wafer transfer device 140 with inert gas (e.g. nitrogen) to reduce wafer oxidation and maintain a dry state and stabilize an internal environment of the wafer transfer device 140.

The wafer transfer device 140 includes a front opening unified pod (FOUP) and a front opening shipping box (FOSB). Detailed improvement design of the wafer transfer device 140 of the present disclosure will be introduced in following paragraphs.

From a beginning of production to packaging and shipment of conventional wafers in the production line, wafers are transported by conventional wafer transport boxes (e.g.: front opening unified pods (FOUP) and front opening shipping boxes (FOSB)). In a semiconductor manufacturing process, there are strict requirements for water vapor or airborne molecular contamination in wafer transport boxes, which must be controlled within a certain range so as not to affect wafers. A current practice is to fill wafer transport boxes with inert gas (nitrogen) to reduce oxidation condition in wafer transport boxes and maintain a dry and stable space of wafer transport boxes.

However, materials used in multiple wafer transport boxes will deteriorate over time or wafer transport boxes have other factors that cause inert gas to escape early. An environment inside a wafer transport box cannot be effectively maintained stable, thereby affecting a yield of wafers or even causing wafers to be scrapped. Therefore, an inability to instantly grasp gas concentration status in a wafer transport box will increase a scrap rate of the wafers. The present disclosure will describe how to improve the above problems in following paragraphs.

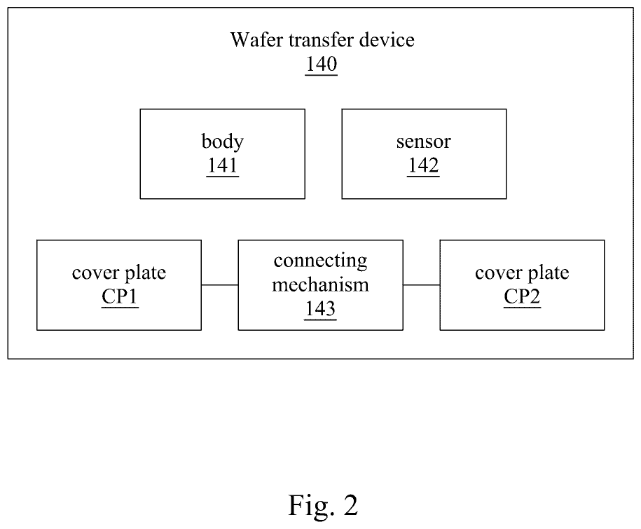

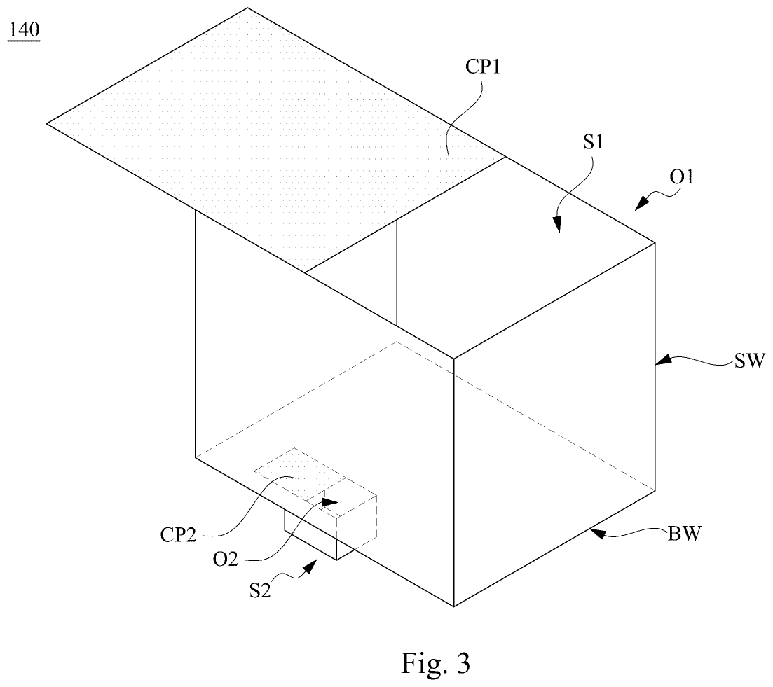

In order to facilitate the understanding a structure of the wafer transfer device 140 of the present disclosure, please refer to FIG. 2 and FIG. 3. FIG. 2 depicts a schematic diagram of the wafer transfer device 140 according to some embodiments of the present disclosure. FIG. 3 depicts a schematic diagram of a structure of the wafer transfer device 140 according to some embodiments of the present disclosure. In some embodiments, please refer to FIG. 2 and FIG. 3, the wafer transfer device 140 includes a body 141, a sensor 142, a connecting mechanism 143, a cover plate CP1 and a cover plate CP2.

In some embodiments, please refer to FIG. 2 and FIG. 3, the body 141 includes a medial wall SW, a bottom wall BW, an opening O1, an accommodation space S1, an accommodation space S2. The opening O1 is relative to the bottom wall BW.

In some embodiments, the accommodation space S1 is defined by the medial wall SW, the bottom wall BW and the opening O1. In some embodiments, the medial wall SW can be four walls or a circular wall. The accommodation space S1 is configured to accommodate wafers and a wafer holder (not shown in the figure). A shape of the medial wall SW can be designed according to actual needs, and is not limited by this embodiment.

In some embodiments, the accommodation space S2 is disposed in one of the medial wall SW and the bottom wall BW. In some embodiments, the accommodation space S2 is disposed in the medial wall SW. In some embodiments, the accommodation space S2 is disposed in the bottom wall BW. The accommodation space S2 includes an opening O2. The opening O2 is configured to connect the accommodation space S1 and the accommodation space S2.

In some embodiments, the cover plate CP1 is configured to cover the opening O1 of the wafer transfer device 140 which is relative to the bottom wall BW to isolate the accommodation space S1 from an environment space outside the wafer transfer device 140.

In some embodiments, the cover plate CP2 is configured to cover the opening O2 of the accommodation space S2 to isolate the accommodation space S2 from the accommodation space S1. In some embodiments, the cover plate CP2 is hidden in one of the medial wall SW and the bottom wall BW so as to avoid collision or friction with the wafer and wafer holder when opening and closing. It should be noted that when the cover plate CP2 is closed, the cover plate CP2 of the of the accommodation space S2 must be at the same height as the bottom wall BW of the wafer transfer device 140 and must not cause a bulge on the bottom wall BW of the wafer transfer device 140. In addition, when the cover plate CP2 is opened, the cover plate CP2 will be only moved a small distance to partially open the opening O2.

It should be further noted that a location and a number of the cover plate CP2 and the accommodation space S2 can be designed according to actual needs, and is not limited by this embodiment. For example, two accommodation spaces S2 can be designed to be located on the medial wall SW and the bottom wall BW respectively. Or, both two accommodation spaces can be designed on the same wall (e.g.: the medial wall SW or the bottom wall BW).

In some embodiments, a material of each of the body 141, the cover plate CP1 and the cover plate CP2 are the same.

In some embodiments, please refer to FIG. 2 and FIG. 3, the sensors 142 are disposed in the accommodation space S2. In some embodiments, the sensors 142 include a gaseous molecular pollutant sensor and a humidity sensor. The sensors 142 are configured to detect a gas concentration of the accommodation space S1 and the accommodation space S1 during a transportation stage of the process. In some embodiments, the gas concentration includes a gas molecular pollutant concentration and humidity.

If the gas concentration of the wafer transfer device 140 is higher than a preset gas concentration, each of the sensors 142 is configured to generate a wireless communication signal. In some embodiments, the wireless communication signal includes ultra-high frequency (UHF) radio frequency identification (RFID) signals. In some embodiments, each of the sensors 142 further includes wireless communication tags. For example, ultra-high frequency (UHF) radio frequency identification (RFID) tags. In some embodiments, the wireless communication tags are integrated in each of the sensors 142.

A number of the sensors 142 can be designed according to actual needs, and is not limited by this embodiment. For example, one of the sensors 142 is a gaseous molecular pollutant sensor, and another of the sensors 142 is a humidity sensor. The two different sensors can be disposed in the aforementioned two accommodation spaces S2 respectively. The two different sensors can be integrated into one sensor module, and the sensor module can be disposed in the accommodation spaces S2.

In some embodiments, the connecting mechanism 143 is coupled to the cover plate CP1 and the cover plate CP2. The connecting mechanism 143 is configured to control the cover plate CP2.

When the cover plate CP1 is configured to cover the opening of the wafer transfer device 140, the connecting mechanism is configured to control the cover plate CP2 to open the opening O2 of the accommodation spaces S2 so as to connect the accommodation space S1 and the second accommodation space S2. When the cover plate CP1 is removed, the connecting mechanism 143 is configured to control the cover plate CP2 to cover the opening O2 of the accommodation space S2 to isolate the accommodation space S2 from the accommodation space S1.

The connecting mechanism 143 can be implemented as a rod composite structure with a latch and a spring structure in the medial wall SW and the bottom wall BW to avoid affecting the wafer and wafer holder of the accommodation space S1 of the wafer transfer device 140. It should be noted that structural components of the connecting mechanism 143 can be designed according to actual needs, and is not limited by this embodiment.

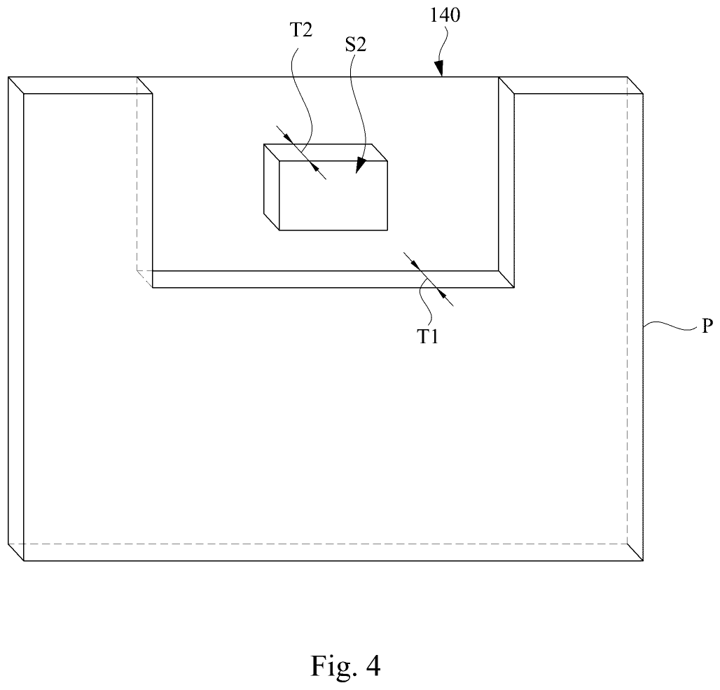

FIG. 4 depicts a schematic diagram of an external bottom view of the wafer transfer device 140 and a transport plate P according to some embodiments of the present disclosure. In some embodiments, please refer to FIG. 3 and FIG. 4, the wafer transfer device 140 is placed on the transport plate P to facilitate transportation during the process. A shape of the transport plate P can be designed according to actual needs, and is not limited by this embodiment. A thickness T2 of the accommodation space S2 of the wafer transfer device 140 does not exceed a thickness T1 of the transport plate P. In other word, the thickness T2 is between the wafer transfer device 140 and the transfer plate P. The thickness T2 can be adjusted according to actual needs.

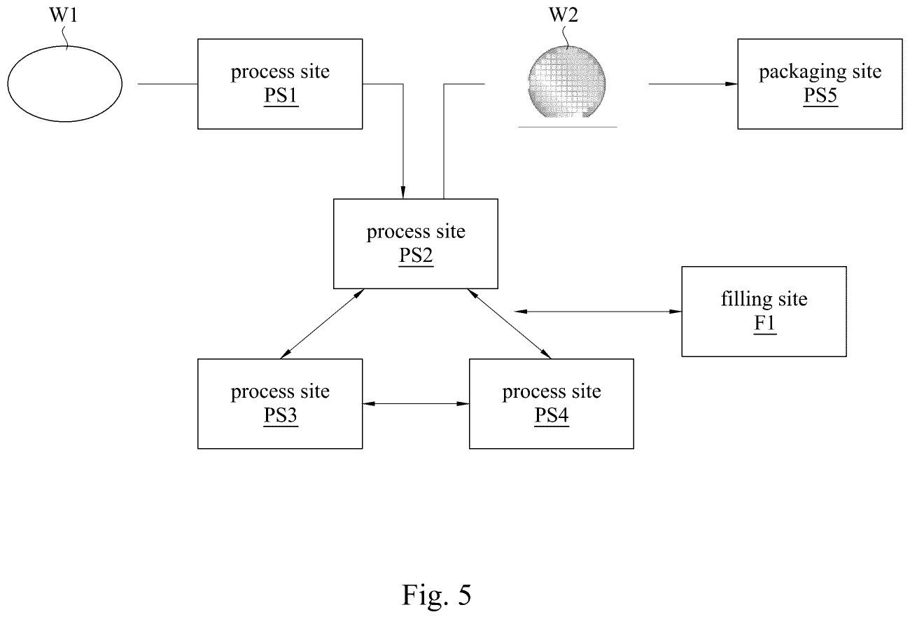

FIG. 5 depicts a schematic flow diagram of a process according to some embodiments of the present disclosure. The schematic flow diagram of the process of in FIG. 5 is a diagram of converting a raw wafer W1 into a wafer W2 after processing. The process includes process sites PS1-PS4, a packaging site PS5 and a filling site F1. In some embodiments, the process site PS1 includes instruments configured to perform different epitaxy procedures. The process site PS2 includes instruments configured to perform different deposition procedures. The process site PS3 includes instruments configured to perform different etching procedures. The process site PS4 includes instruments configured to perform different photolithography procedures. The packaging site PS5 instruments configured to perform packaging procedures. The filing device in FIG. 1 can be disposed in the filling site F1 and the packaging site PS5.

It should be noted that the raw wafer W1 must go through dozens or hundreds of different processes (e.g.: different deposition procedures, different etching procedures and different photolithography procedures) to produce the wafer W2. A number of each of the process sites is not limited by this embodiment.

In some embodiments, please refer to FIG. 1 and FIG. 5, each of the detectors 120 is disposed at the instruments of the process sites PS1-PS4 and the packaging site PS5. In some embodiments, each of the detectors 120 is disposed between the instruments of the process sites PS2-PS4 to instantly read the gas concentration of the wafer transfer device 140. For example, each of the detectors 120 is next to tracks of an overhead hoist transfer system (not shown in the figure).

In some embodiments, please refer FIG. 1, FIG. 2 and FIG. 5, if the gas concentration of each of the wafer transfer devices 140 is higher than the preset gas concentration, the sensors 142 of each of the wafer transfer devices 140 is configured to generate the wireless communication signal to spread the wireless communication signal around. When the detectors 120 are configured to receive the wireless communication signal from the sensors 142 of each of the wafer transfer devices 140, the detectors 120 are configured to notify the control center 110 so that the control center 110 is configured to transmit the wafer transfer device 140 to the filling device 130 at the filling site F1 from the process.

In other words, the control center 110 is further configured to record a next process of the wafer transfer device 140 and interrupt a transportation process of the process so as to transfer the wafer transfer device 140 to the filling device 130 at the filling site F1 (or regarded as be transported to a sub-route to the wafer transfer device 140 shown in the FIG. 5) to perform the filling procedure. A function of the filling procedure is to adjust the gas concentration of the wafer transfer device 140 to drop to a target gas concentration with inert gas (nitrogen). After the filling procedure, the wafer transfer device 140 is retransmitted back the next process recorded by the control center 110.

It should be noted that the cover plate CP2 is configured to covert the opening O2 of the accommodation space S2 to isolate the accommodation space S2 from the accommodation space S1 to achieve a purpose of protecting the sensors 142 of the wafer transfer device 140.

Based on the aforementioned embodiments, the present disclosure provides a wafer transfer device detection system and a wafer transfer device. Through a wafer transfer device detection system and a wafer transfer device designed in the present disclosure, a gas concentration (e.g.: a gas molecular pollutant concentration and humidity) inside a wafer transfer device can be visualized, and a gas concentration of a wafer transfer device can be immediately adjusted to an appropriate gas concentration to maintain a stability of an internal environment of a transfer device at all times and is not affected by aging of a wafer transfer device or other factors. It can also be used to monitor a humidity status in wafer transfer device in real time before wafers are loaded for packaging and shipment, and fill it with inert gas (nitrogen) appropriately to avoid affecting wafers due to a humidity of the wafer transfer device, resulting in poor product yield or even scrapping.

Certain terms are used in the specification and the claims to refer to specific components. However, those of ordinary skill in the art would understand that the same components may be referred to by different terms. The specification and claims do not use the differences in terms as a way to distinguish components, but the differences in functions of the components are used as a basis for distinguishing. Furthermore, it should be understood that the term “comprising” used in the specification and claims is open-ended, that is, including but not limited to. In addition, “coupling” herein includes any direct and indirect connection means. Therefore, if it is described that the first component is coupled to the second component, it means that the first component can be directly connected to the second component through electrical connection or signal connections including wireless transmission, optical transmission, and the like, or the first component is indirectly electrically or signally connected to the second component through other component(s) or connection means.

It will be understood that, in the description herein and throughout the claims that follow, the phrase “and/or” includes any and all combinations of one or more of the associated listed items. Unless the context clearly dictates otherwise, the singular terms used herein include plural referents.

Although the present disclosure has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present disclosure without departing from the scope or spirit of the present disclosure. In view of the foregoing, it is intended that the present disclosure cover modifications and variations of this disclosure provided they fall within the scope of the following claims and their equivalents.

What is claimed is:

1. A wafer transfer device detection system, comprising:
a plurality of detectors, disposed between a plurality of process equipment in a process;
a filling device, configured to perform a filling procedure;
a wafer transfer device, comprising a body and a sensor disposed in the body, wherein the wafer transfer device is configured to transmit a wafer of the process, wherein the sensor is configured to detect a gas concentration inside the wafer transfer device during a transportation stage of the process, wherein if the gas concentration is higher than a preset gas concentration, the sensor is configured to generate a wireless communication signal; and
a control center, coupled to the detectors and the filling device, wherein if the detectors are configured to receive the wireless communication signal, the detectors are configured to notify the control center so that the control center is configured to transmit the wafer transfer device to the filling device from the process, wherein the control center is configured to control the filling device to perform the filling procedure so as to adjust the gas concentration to a target gas concentration to retransmit the wafer transfer device back to the process.

2. The wafer transfer device detection system of claim 1, wherein the body of the wafer transfer device comprises:
a medial wall;
a bottom wall;
a first opening;
a first accommodation space, jointly defined by the medial wall, the bottom wall and the first opening, wherein the first accommodation space is configured to accommodate the wafer and a wafer holder; and
a second accommodation space, disposed in one of the medial wall and the bottom wall, wherein the second accommodation space comprises a second opening.

3. The wafer transfer device detection system of claim 2, wherein the sensor is disposed in the second accommodation space, and is configured to detect the gas concentration of the first accommodation space and the second accommodation space.

4. The wafer transfer device detection system of claim 2, wherein the body of the wafer transfer device further comprises:
a first cover plate, configured to cover the first opening of the wafer transfer device to isolate the first accommodation space from an environment space outside the wafer transfer device.

5. The wafer transfer device detection system of claim 4, wherein the body of the wafer transfer device further comprises:
a second cover plate, configured to cover the second opening to isolate the second accommodation space from the first accommodation space.

6. The wafer transfer device detection system of claim 5, wherein the body of the wafer transfer device further comprises:
a connecting mechanism, coupled to the first cover plate and the second cover plate, wherein the connecting mechanism is configured to control the second cover plate, wherein when the first cover plate is configured to cover the first opening, the connecting mechanism is configured to control the second cover plate to open the second opening so as to connect the first accommodation space and the second accommodation space.

7. The wafer transfer device detection system of claim 6, wherein the first cover plate is removed, the connecting mechanism is configured to control the second cover plate to cover the second opening to isolate the second accommodation space from the first accommodation space.

8. The wafer transfer device detection system of claim 5, wherein the second cover plate is hidden in the one of the medial wall and the bottom wall.

9. The wafer transfer device detection system of claim 1, wherein the sensor comprises a gaseous molecular pollutant sensor and a humidity sensor.

10. A wafer transfer device, configured to transmit a wafer of a process, wherein the wafer transfer device comprises:
a body, comprising:
a medial wall;
a bottom wall;
a first opening relative to the bottom wall;
a first accommodation space, jointly defined by the medial wall, the bottom wall and the first opening, wherein the first accommodation space is configured to accommodate the wafer and a wafer holder; and
a second accommodation space, disposed in one of the medial wall and the medial wall, wherein the second accommodation space comprises a second opening; and
a sensor, disposed in the second accommodation space, and configured to detect a gas concentration of the first accommodation space and the second accommodation space during a transportation stage of the process, wherein if the gas concentration is higher than a preset gas concentration, the sensor is configured to generate a wireless communication signal to notify a control center to transmit the wafer transfer device to a filling device so that the filling device is configured to perform a filling procedure so as to adjust the gas concentration to a target gas concentration.

11. The wafer transfer device of claim 10, further comprising:
a first cover plate, configured to cover the first opening of the wafer transfer device to isolate the first accommodation space from an environment space outside the wafer transfer device.

12. The wafer transfer device of claim 11, further comprising:
a second cover plate, configured to cover the second opening to isolate the second accommodation space from the first accommodation space.

13. The wafer transfer device of claim 12, further comprising:
a connecting mechanism, coupled to control the second cover plate, wherein when the first cover plate is configured to cover the first opening, the connecting mechanism is configured to control the second cover plate to open the second opening so as to connect the first accommodation space and the second accommodation space.

14. The wafer transfer device of claim 13, wherein when the first cover plate is removed, the connecting mechanism is configured to control the second cover plate to cover the second opening to isolate the second accommodation space from the first accommodation space.

15. The wafer transfer device of claim 12, wherein the second cover plate is hidden in the one of the medial wall and the medial wall.
